(19) United States
(12) Patent Application Publication
(10) Pub. No.: US 2006/0275223 A1
(43) Pub. Date: Dec. 7, 2006

(54) Erythritol compositions for teeth and gums

(76) Inventor: James B. Burr, Provo, UT (US)

Correspondence Address: WORKMAN NYDEGGER;(F/K/A WORKMAN NYDEGGER & SEELEY), 60 EAST SOUTH TEMPLE, 1000 EAGLE GATE TOWER, SALT LAKE CITY, UT, 84111, US

(21) Appl. No.: 11/185,345

(22) Filed: Jul. 20, 2005

Related U.S. Application Data
(63) Continuation-in-part of application No. 11/143,146, filed on Jun. 2, 2005.

(51) Int. Cl. A61K 8/34

(52) U.S. Cl. 424/49

(57) ABSTRACT

An oral hygiene composition for use in cleansing a subject's teeth and gingiva can include an effective amount of erythritol admixed with an acceptable carrier. The oral hygiene composition can be configured into a gel or paste that is capable of being applied to one of a subject's tooth and gingiva in a sufficient amount so as to be retained thereto for a duration adequate for cleansing the subject's teeth and gingiva. Additionally, a method of oral cleansing in a subject incapable of adequately self-performing dental hygiene techniques can include administering the oral hygiene composition to one of the subject's tooth and gingiva. The method can also include retaining the oral hygiene composition to the one of the tooth and gingiva for a sufficient duration to effect the oral cleansing.

### CROSS-REFERENCE TO RELATED APPLICATIONS

This application claims priority to a U.S. patent application Ser. No. 11/143,146, filed Jun. 2, 2005, attorney docket no. 16107.3, entitled, “Erythritol Compositions for Nasopharynx Cleansing,” with James B. Burr as inventor, which is incorporated herein by reference in its entirety.

### BACKGROUND OF THE INVENTION

1. The Field of the Invention

The present invention relates to oral hygiene compositions and methods of use. More particularly, this invention relates to erythritol-containing oral hygiene compositions that can be used to clean the tooth and gums, and treat and/or prevent dental caries caused by bacteria.

2. The Relevant Technology

The temperature and wet conditions of the mouth as well as the abundant supply of food particles provides an almost ideal habitat for bacteria to grow. As a consequence, bacteria such as, for example, streptococcus mutans are often found to be present in a typical subject's mouth. The strep mutans use sugars and other carbohydrates for sustenance, which can be found in the mouth in high quantities, especially after the subject eats a meal. These bacteria have been found to be at least partially responsible for the formation of dental caries because they produce and release acid into the mouth around the teeth. This can lead to the formation of cavities in the subject's teeth by the acid reacting with and dissolving the protective enamel layer. Also, the presence of strep mutans can lead to other unfavorable health complications.

Recently, erythritol has been shown to be effective in inhibiting bacteria growth. Erythritol is a polyol that occurs naturally in a wide variety of fruits and vegetables, and has functional properties similar to sucrose, though its caloric content is close to zero. Also, erythritol does not cause digestive intolerance, unlike many other polyol sweeteners. Although erythritol is chemically a polyol, it is such a small molecule that it behaves very differently from other polyols in the way it passes through the digestive system. The proximal intestine absorbs these types of molecules at a rate related to their molecular size. Therefore, erythritol, a four carbon sugar substitute, is absorbed at a much faster rate than other sugars with larger molecules such as mannitol or glucose. Some larger polyols have slow rates of absorption from the intestine, which leads to osmotic diarrhea. Studies have indicated that erythritol, even when it comprises 20% of the diet, does not cause diarrhea. Additionally, because of erythritol's low glycemic index, it does not affect blood glucose levels and so can safely be consumed by people with diabetes.

These unique properties have resulted in erythritol being used as a sugar replacement, and the FDA has awarded erythritol GRAS (generally recognized as safe) status. Erythritol is currently used as a sugar replacement in candies, syrups, low calorie beverages, and is increasingly being used in the pharmaceutical and dental industries. Erythritol does not promote tooth decay because bacteria cannot metabolize it. In fact, erythritol is capable of partially inhibiting oral bacteria in their ability to ferment certain sugars.

Thus, what is needed is an erythritol composition configured for being administered to the teeth and gingiva of infants and toddlers. Also, it would be beneficial to have methods of administering erythritol for cleansing and anti-caries purposes.

### BRIEF SUMMARY OF EMBODIMENTS OF THE INVENTION

An embodiment of the present invention is an oral hygiene composition for use in cleansing a subject's teeth and gums, where the subject can be an infant or toddler. The oral hygiene composition includes an effective amount of erythritol. Also, the opal hygiene composition includes an acceptable carrier admixed with the erythritol. The acceptable carrier can be configured into a gel or paste that is capable of being applied to at least one of a subject's tooth or gingiva in a sufficient amount so as to be retained thereto long enough to cleanse the teeth and gingiva. As such, the composition can be applied to teeth and/or gingiva so that it sticks thereto, which allows the erythritol to be absorbed into the gingiva and/or dispersed around the teeth.

Another embodiment of the present invention is a method of oral cleansing in a subject incapable of self-performing dental hygiene techniques. Such a subject can include infants, toddlers, and children that are not capable of brushing their teeth. The method includes administering an oral hygiene composition to at least one of the subject's tooth or gingiva in an effective quantity. The oral hygiene composition contains an effective amount of erythritol admixed into an acceptable carrier. The method also includes retaining the oral hygiene composition to the tooth and/or gingiva for a sufficient duration. This allows the composition to stick to the teeth and gingiva so that the erythritol is able to be absorbed into the gingiva and dispersed around the teeth.

Another embodiment of the present invention is a method of treating and/or preventing dental caries in a child. The method can include the administering the erythritol-containing oral hygiene composition to at least one of a tooth or gingiva of the child, and retaining the composition thereto for a sufficient duration. Also, the method can include inhibiting one of bacteria acid production and bacteria growth. Additionally, the method can include inhibiting the formation of dental caries.

These and other embodiments and features of the present invention will become more fully apparent from the following description and appended claims, or may be learned by the practice of the invention as set forth hereinafter.

### DETAILED DESCRIPTION OF EMBODIMENTS OF THE INVENTION

Embodiments of the present invention include teeth and gum cleansing compositions, and associated methods of using the compositions. As the present invention is disclosed and described, it is to be understood that this invention is not limited to the particular materials, combinations, and methods disclosed herein. Accordingly, this disclosure is extended to equivalents of the materials, combinations, and methods as would be recognized by one of ordinary skill in the relevant arts. It should be understood that terminology employed herein is used for the purpose of describing particular embodiments only and is not intended to be limiting.

As used herein, the term “erythritol” refers to “erythritol” or amounts of “erythrose” that can convert to erythritol. Also, the term can include “erythritol and erythrose” as well as the various isomeric forms of erythritol or erythrose. Erythritol, erythrose, and mixtures of erythritol and erythrose can be equivalent, and all equally effective in equal amounts in all therapeutic uses.

As used herein, the term “non-cariogenic” refers to sweeteners which are not able to be metabolized by oral microbes and therefore do not contribute to the formation of dental caries. For example, erythritol is a non-cariogenic sweetener.

As used herein, the terms “teeth” or “tooth” is meant to include natural teeth, and any other hard dental surface, such as crowns, caps, fillings, bridges, dental implants, dental restorations, and the like, that are permanently fixed within the oral cavity and cleansed in situ within the oral cavity.

As used herein, the term “dentifrice” is meant to include a gel or paste formulation that is used to clean the teeth and/or gingiva.

As used herein, the term “acceptable carrier” is meant to refer to any safe and effective materials for use in the compositions of the present invention that can carry and deliver erythritol. Such materials can include polysaccharides, acidic compounds, buffering agents, abrasive polishing materials, alkali metal bicarbonate salts, thickeners, humectants, water, surfactants, opacifiers such as titanium dioxide, flavorants, sweetening agents, coloring agents, and the like.

As used herein, the term “flavoring agent” or “flavorants” is meant to refer to flavor essences and equivalent synthetic materials which are added to flavor the composition. The flavoring agent can also include specific materials which are added to provide a warming or cooling sensation.

As used herein, “permeation enhancer,” “penetration enhancer,” “chemical enhancer,” “enhancer” or similar terms refers to compounds and mixtures of compounds that enhance the permeation of a drug across the skin, gingiva, or buccal mucosa.

As used herein, the terms “tartar,” and “calculus” are used interchangeably and refer to mineralized dental biofilms that form on the outer surface of teeth.

As used herein, the term “plaque” is used to refer to a film of mucus and bacteria on a tooth surface.

As used herein, the term “effective amount” refers to the minimal amount through the maximal amount of a substance or agent, which is sufficient to achieve a desired effect. For example, an effective amount of erythritol in a dental composition would be the minimum amount that provides the desired physiological effect such as cleansed teeth, inhibited bacteria growth, inhibited bacteria production, reduced inflammation and/or soreness during teething. Accordingly, an “effective amount” should include a safe and effective amount, which is meant to include an amount of an agent (e.g., erythritol) high enough to significantly improve the condition to be treated, but low enough to avoid serious side effects (at a reasonable benefit/risk ratio), within the scope of sound medical/dental judgment. The safe and effective amount of the agent may vary with the particular condition being treated, the age and physical condition of the patient being treated, the severity of the condition, the duration of treatment, the nature of concurrent therapy, the specific form of the source employed, and the particular carrier from which the agent is applied.

As used herein, the term “container” as described herein, means a jar, cup, can, tube, tub, pump, bottle or any other composition holding or dispensing means.

As used herein, the term “unit dose form” refers to physically discrete units suitable as unitary dosages for human subjects and other mammals, each containing a predetermined quantity of active material calculated to produce the desired therapeutic effect.

It should be recognized that the chemical nomenclature employed herein is used for convenience and brevity, and any of the elements recited additionally includes the monovalent or multivalent ions thereof. For example, zinc (Zn) includes the element and an ion of any valence, if the valence is possible.

Concentrations, amounts, particles sizes and other numerical data may be presented in a range format. It is to be understood that such a range format is used merely for convenience and brevity and should be interpreted flexibly to include not only the numerical values explicitly recited as the limits of the ranges, but also to include all the individual numerical values or sub-ranges encompassed within that range as if each numerical value and sub-range is explicitly recited. For example, the particle diameter of the abrasive agent can range of about 0.1 to about 50 microns should be interpreted to include not only the explicitly recited limits, but also to include sub-ranges such as about 5 to about 15 microns as well as other sub-ranges. Also, the enumeration of such ranges should be interpreted to include such individual diameters such as 1, 2, 10, and 20 micrometers as well as sub-ranges between these individual diameters. This interpretation should apply regardless of the breadth of the range or the characteristic being described, and should apply to ranges having both upper and lower numerical values as well as open-ended ranges reciting only one numerical value.

As used herein, percentages and ratios are by weight of total composition, unless otherwise indicated.

Additionally, the oral hygiene composition is described herein by enumeration of various examples of components as well as various examples of component concentrations. However, it should be recognized that these are merely illustrated for example purposes and should not be interpreted to limit the components to the explicitly enumerated examples, where the examples should be extended to equivalents in the art known to have similar functionalities. Also, it should be recognized that the concentrations of these components are given only for example, and the actual concentration of any of the components can vary from the values exemplified because the specific components and associated concentrations within a composition can vary the amounts of the other components. Thus, the exemplified components and concentrations are provided only as indicators of the types and approximate amounts of components that can be included within the oral hygiene compositions of the present invention.

The oral hygiene composition may be a single-phase oral composition or may be a combination of two or more oral compositions delivered in various phases. The oral hygiene composition is a product that, in the ordinary course of usage, is not intentionally swallowed for purposes of systemic administration of particular therapeutic agents, but rather, is retained in the oral cavity on the teeth and gingiva for a time sufficient to release erythritol into the mouth so that it can be absorbed into the gingiva as well as disperse around the teeth. Additionally, in one embodiment the oral dentifrice in accordance with the present invention is not a chewing gum because toddlers and infants are incapable of properly chewing or using such gums. Also, it may be beneficial for the oral hygiene composition to be a gel or paste so that it can be retained on the teeth or gingiva for a sufficient time for the erythritol to come into contact with substantially all of the dental surfaces and/or oral tissues for cleansing the oral cavity. Moreover, it can be preferred that the gel or paste is configured to provide a beneficial effect without brushing.

In accordance with the foregoing, the present invention provides an infant and/or toddler oral hygiene composition or dentifrice intended for cleaning the gum tissue (e.g., gingiva) and teeth. Also, the oral hygiene composition can reduce gum inflammation and dental caries during and after teething. Accordingly, the oral hygiene composition can contain erythritol, a thickening agent (e.g., carboxymethylcellulose), a calcium agent (e.g., calcium glycerol phosphate), artificial flavoring (e.g., mint or lemon), and a preservative (e.g., grapefruit seed extract). While a erythritol-containing composition is ingestible, some embodiments are oral-compatible so as to not alter the microflora balance of the oral cavity.

An embodiment of the present invention is an oral hygiene composition for use in treating and/or preventing dental caries in a subject. Also, the oral hygiene composition can be used to provide a medium for frequent cleansing of the oral cavity to eliminate the bacterial film that collects on infant and toddler teeth and gingiva. Additionally, the oral hygiene composition can be used to reduce the inflammation and soreness that regularly occurs during the eruption of infant teeth, which is also known as teething. Further, the oral hygiene composition can be used to clean infant and toddler baby teeth prior to an age when they can adequately perform dental hygiene techniques such as brushing. Furthermore, the oral hygiene composition can be used to decrease the amount and negative consequences of oral bacterial during and after the teething in order to minimize the formation of dental caries on baby teeth. Still further, the oral hygiene composition can be used to aid in naturally inducing a remineralization process of the tooth enamel. Still yet a furthermore, the oral hygiene composition can be used to naturally decrease the decalcification on a tooth's surface.

Erythritol provides a therapeutic effect, such as an antibacterial or anti-caries effect. Erythritol can prevent the formation of dental caries by inhibiting the production of acid by bacteria and/or inhibiting the growth of bacteria in the mouth. The oral hygiene composition includes erythritol at a concentration of from about 1% to about 80%, in one embodiment from about 10% to about 65%, and in another embodiment from about 10% to about 50% by weight.

In any event, the composition can include an effective amount of erythritol. Also, the composition includes an acceptable carrier admixed with the effective amount of erythritol. As such, the composition can be configured into an oral hygiene composition that is capable of being applied to one of a subject's tooth and gingiva in a sufficient amount. The sufficient amount is a quantity that provides for at least a portion of the amount applied to be retained on the tooth and gingiva for a duration adequate for the uses described herein. Also, the composition can be dehydrated or in a concentrated composition that is formulated to be diluted or mixed with water to provide the proper gel, paste, or other characteristics to retain the erythritol to the teeth and/or gingiva.

In order to enhance a gelatinous or tooth-retention characteristic of the oral hygiene composition, the composition can include a thickening material or binders to ensure that the final composition has the desired consistency. The thickener can also be considered to be a viscosity increaser because the viscosity of the composition can be changed upon the addition of the thickener which can result in the formation of a gel. Various polymers are representative of the viscosity increaser. Such polymers include carboxyvinyl polymers, polyhydroxyacids, alginates, polyacrylic acids, pentosans, polysulfates, polyorthoesters, and polysaccharides. More particularly, viscosity increasing polysaccharides can include homopolysaccharides such as celluloses, amyloses, inulins, chitins, chitosans, amylopectins, glycogens, and pectins. Also, viscosity increasing polysaccharides can include heteropolysaccharides such as hemicelluloses, glucomannans, galactoglucomannans, xyloglucans, methylglucuronoxylans, arabinoxylans, methylglucuronoarabinoxylans, glycosaminoglycans, chondroitins, hyaluronic acids, and alginic acids. Some more specific examples of thickening agents include, carrageens, carboxymethylcellulose, hydroxyethyl cellulose, and water soluble salts of cellulose ethers such as sodium carboxymethylcellulose and sodium hydroxyethyl cellulose.

Also, natural gums such as gum karaya, xanthum gum, gum arabic, and gum tragacanth can be used as part of the thickening agent to further improve the texture. Specific examples of thickening gum components can include sodium alginate, ammonium alginate, sodium calcium alginate, calcium alginate, potassium alginate, esters of alginic acid like acetyl esters, ethyl esters, propyl esters, butyl esters, pentyl esters, hexyl esters, heptyl esters, octyl esters, nonyl esters, decyl esters, and propyl glycerol alginate. Thickening agents such as viscosity increasers can be used in an amount, for example, at from about 1% to about 50% by weight of the composition. More preferably, 5% to 30%, and most preferably about 8% to 15% by weight. However, the amount of thickener can vary depending on the other constituents in the composition.

Plasticizers can also be combined into composition to aid in providing an adequate gelatinous consistency, which may also affect the viscosity of the gel. Some examples of plasticizers include glyceryl triacetate, acetylated monoglyceride, glyceryl tributyrate, ethyl laurate, ethyl acetoacetate, diethyl tartrate, ethyl or butyl lactates, diethyl malate, ethyl oleate, castor oil, succinylated monoglycerides and the like. Oral hygiene gel compositions can include up to about 10%, preferably from about 0.1% to about 3%, by weight of plasticizer.

The oral hygiene composition can also include a calcium agent. Calcium agents can be used to provide the composition with various characteristics, as well as provide calcium for remineralization of the dental enamel. The calcium agent can be selected from, for example, calcium acetate, calcium citrate, calcium gluconate, calcium glycerol phosphate mono-calcium phosphate, mono-calcium phosphate anhydrous, di- and tri-calcium phosphate, calcium sulfate, calcium carbonate, calcium lactate, calcium phosphate, and the like. The calcium agent can be present at from about 1% to about 15%, more preferably about 2.5% to about 10%, and most preferably about 4% to about 6% by weight.

In addition, an oral hygiene composition in accordance with the present invention can include anti-plague agents. Anti-plaque agents can include any substances which inhibit the accumulation of bacterial deposits on the surfaces of the oral cavity such as fluoride ion sources. When combined with erythritol, such anti-plaque agents can cooperate to reduce cavities because fluoride ion sources can act to protect teeth against the formation of dental caries and decay. A wide variety of fluoride ion yielding materials can be employed as sources of fluoride in the present compositions. Examples of suitable fluoride ion yielding materials for use in the present invention include sodium fluoride, potassium fluoride, stannous fluoride, ammonium fluoride and mixtures thereof. Sodium fluoride is particularly preferred. Preferably, the present composition can include from about 50 ppm to about 10,000 ppm, and more preferably from about 100 ppm to about 3000 ppm of fluoride ions.

Desensitizing agents can also be combined with the oral hygiene composition of the present invention. Desensitizing agents, which can be referred to as pain-relieving agents, may include, but are not limited to, strontium chloride, and potassium nitrate. Additionally, the desensitizing agents can be analgesics such as, for example, the non-steroidal anti-inflammatory agents ketorolac, flurbinprofen, ibuprofen, naproxen, indomethacin, aspirin, ketoprofen, piroxicam, meclofenamic acid, and the like.

Oral malodor control agents can also be included in the oral hygiene composition of the present invention. Oral malodor control agents include a wide variety of materials such as, for example, 5-chloro-2-(2,4-dichlorophenoxy)-phenol (“triclosan”), phthalic acid and its salts, magnesium mono-potassium phthalate, chlorhexidine, alexidine, hexetidine, sanguinarine, benzalkonium chloride, salicylanilide, domiphen bromide, cetylpyridinium chloride (“CPC”), tetradecylpyridinium chloride (“TPC”), N-tetradecyl-4-ethylpyridinium chloride (“TDEPC”), octenifine, delmopinol, octapinol, and piperidine derivatives, zinc/stannous ion agents, antibiotics, augmentin, amoxicilline, tetracycline, doxycycline, minocycline, and metronidazole, methyl salicyclate, and the like.

Another class of oral malodor control agents includes absorbent materials, which can absorb odor-causing substances therein. As such, the absorbent material is used to absorb, bind or otherwise complex with volatile oral malodor substances. Examples of such absorbent materials include talc, zeolite, cyclodextrin, silica shell, and the like. Such materials are can be used at a concentration of from about 0.5% to about 10%, and preferably from about 1% to about 5% by weight of the composition.

Additionally, a wide variety of salts can be included in the present invention. These salts can include “water-insoluble salts” having a solubility of less than about 0.5 g per 100 ml at 25 ° C, and “water soluble salts” having a solubility of greater than or equal to about 0.5 g per 100 ml at 25 ° C. It is also possible to use mixtures of these Such mixtures can have several advantages in the compositions of the present invention because they are likely to have different complexing properties with anions such as phosphate anions. In addition, the salts have different release rates in the saliva and can therefore act to provide controlled release profiles. Some examples of salts that are suitable for use in the oral hygiene composition include acetates, ammonium sulfates, bromides, chlorides, chromates, citrates, dithionates, fluorosilicates, tartrates, fluorides, formates, iodides, nitrates, phenol sulfates, salicyclates, gluconates, succinates, glycerophosphates, lactates, and the like.

The oral hygiene composition can also include water. When water is used in the present invention, the water preferably is of low ion content and free of organic impurities. The amount of water in a composition should be considered to be not only the amount added as free water, but also water which is introduced with other materials, such as with sorbitol, silica, surfactant, or other components. Water can be included at from about 10% to about 80%, or from about 15% to about 65% by weight of the composition. In some embodiments of the present invention, it may be preferred that the composition include less than about 10%. The low levels of water are can be used when the composition further includes other moisture retaining components such as humectants or solvents, or is designed to be mixed with water to a desirable consistency before being used. Also, low water levels can reduce oxidization or hydrolysis of some of the compositional components.

The present oral hygiene composition can also include surfactants. Suitable surfactants are those which are reasonably stable and foam throughout a wide pH range. The surfactant may be anionic, amphoteric, zwitterionic, cationic, or mixtures thereof. For example, anionic surfactants can include the water soluble salts of alkyl sulfates having from about 8 to about 20 carbon atoms in the alkyl chain (e.g., sodium alkyl a sulfate), and the water soluble salts of monoglycerides sulfonates of fatty acids having from about 8 to about 20 carbon atoms such as sodium lauryl sulfate. Nonionic surfactants can be broadly designed as uncharged surfactants having a hydrophilic portion and a hydrophobic portion. The amphoteric surfactants useful in the present invention can include derivatives of aliphatic secondary and tertiary amines in which the aliphatic hydrocarbon chain can be straight chain or branched, and wherein one of the aliphatic chains contains from about 8 to about 18 carbon atoms. Also, the amphoteric surfactant can include an anionic water solubilizing group such as, for example, carboxylates, sulfonates, sulfates, phosphates, and phosphonates. The oral hygiene composition can include surfactants at from about 0.25% to about 12%, from about 0.5% to about 8%, or from about 1% to about 6% by weight of the composition.

The oral hygiene composition can also include an antioxidant. Antioxidants are generally recognized to include vitamin E, ascorbic acid, uric acid, carotenoids, vitamin A, flavenoids, polyphenols, herbal antioxidants, melatonin, aminoindoles, lipoic acids, and the like.

Coloring substances can also be included in the oral hygiene composition. Specific examples include talc, mica, magnesium carbonate, calcium carbonate, magnesium silicate, aluminum magnesium carbonate, silica, titanium dioxide, zinc oxide, red iron oxide, brown iron oxide, yellow iron oxide, black iron oxide, ferric ammonium ferrocyanide, manganese violet, ultramarine, nylon powder, polyethylene powder, methacrylate powder, polystyrene powder, silk powder, crystalline cellulose, starch, titanated mica, iron oxide titanated mica, bismuth oxychloride, and the like. When used, these substances can be present in the composition at from about 0.05% to about 20%, from about 0.1% to about 15%, or from about 0.25% to about 10% by weight, of the composition. Opacifiers such titanium dioxide may also be added to the composition generally at from about 0.25% to about 5%, by weight of the composition.

The oral hygiene composition can also include materials that remove or bleach stains on or in tooth surfaces. Such substances can include, for example, peroxides, metal chlorites, perborates, percarbonates, peroxyacids, persulfates, and the like. Suitable peroxide compounds include hydrogen peroxide, urea peroxide, calcium peroxide, carbamide peroxide and the like. Suitable metal chlorites include calcium chlorite, barium chlorite, magnesium chlorite, lithium chlorite, sodium chlorite and potassium chlorite. Additional bleaching substances may be hypochlorite, and chlorine dioxide. The concentration of these substances can be dependent on the available oxygen or chlorine. Accordingly, the concentration can range from about 0.1% to about 35%, from about 1% to about 25%, or from about 5% to about 10% by weight of the composition.

Additionally, to maintain a favorable consistency, a humectant can be included. This is because humectants can prevent the composition from hardening upon exposure air. Some examples of humectants can include glycerin, sorbitol, polyethylene glycol, propylene glycol, and other edible polyhydric alcohols. The humectant can be present in an amount of from about 0.5% to about 60%, or from about 15% to about 45% by weight of the composition.

Various oils can also be included in the oral hygiene composition. Some examples of oils can include the hydrogenated vegetable oils such as hydrogenated palm oil, hydrogenated soybean oil, hydrogenated cotton seed oil, and various other hydrogenated vegetable oils. When used, the oils can be included up to about 20% by weight of the composition.

The present invention may also include a bicarbonate salts to buffer the pH of the oral hygiene composition. Bicarbonate salts are soluble in water, and can release carbon dioxide into aqueous systems. Sodium bicarbonate is the preferred bicarbonate. The oral hygiene composition can include a bicarbonate salt at from about 0.5% to about 20% by weight of the composition.

Additionally, the oral hygiene composition may include other buffering agents. Buffering agents can be used to adjust the pH of the compositions of a range of from about pH 3 to about pH 10. Some examples of buffering agents include hydroxides, carbonates, borates, silicates, phosphates, imidazoles, and mixtures thereof. Specific examples of buffering agents include monosodium phosphate, trisodium phosphate, sodium hydroxide, potassium hydroxide, carbonate salts, sodium carbonate, imidazole, pyrophosphate salts, citric acid, and sodium citrate. Buffering agents are used in concentrations at from about 0.1% to about 20% by weight of the composition.

The oral composition may also incorporate an acidic compound, which may be organic or inorganic. The acidic compound may be any material that will act as a proton donor capable of neutralizing a base such as a bicarbonate. Examples of some acidic compounds can include carboxylic acids, phosphoric acids, alpha-hydroxy acids, sulfonic acids, and the like. Specific examples of acids include citric acid, malic acid, alginic acid, succinic acid, lactic acid, tartaric acid, glycolic acid, adipic acid, potassium bitartrate acid, acid sodium citrate, phosphoric acid, boric acid, and acid phosphate pyrophosphate salts. Acid anhydrides and acid salts of the above acids may also be used. The oral hygiene composition can include an acidic compound at from about 0.01% to about 10% by total weight of the composition.

Additionally, the oral hygiene gel of the present invention can include a softener or mixture of softeners which, when incorporated into the gel base, assist in modifying the texture and consistency properties. Suitable softeners include fatty materials such as, for example, lanolin, stearic acid, sodium stearate and potassium stearate; polyhydric alcohols such as glycerine, propylene glycol, and the like. When used, the softener can be included up to about 20%, more preferably from about 0.1% to about 10% by weight of gel.

Also, the oral hygiene composition can include an emulsifier such as glycerol monostearate, lecithin, fatty acid monoglycerides, diglycerides, propylene glycol monostearate and mixtures thereof. When used, the emulsifier can be present up to about 10%, and preferably from about 2% to about 6% by weight of the composition.

Besides erythritol, the oral hygiene composition can include other non-cariogenic sweeteners. Some examples of non-cariogenic sweetening agents that can be used with erythritol include sorbitol, mannitol, maltitol, isomalt, hydrogenated starch hydrolisate, and other non-cariogenic edible polyols such as glycerin and xylitol. Also, a combination of an effective amount of erythritol and xylitol can provide favorable oral cleansing.

High intensity sweeteners can also be used because they are typically non-cariogenic. Some examples of high intensity sweeteners include: dipeptide based sweeteners such as L-aspartyl-L-phenylalanine methyl ester (Aspartame), L-alpha-aspartyl-N-(2,2,4,4-tetramethyl-3-thietanyl)-D-alaninamide hydrate (Alitame), and the like; saccharin and its soluble salts such as sodium or calcium saccharin salts; cyclamate salts; and other high intensity sweeteners. Compositions of the present invention can include from about 0.01% to about 2.0%, or from about 0.05% to about 0.5% by weight of a high intensity sweetener.

The oral hygiene composition can include some limited amount of a cariogenic sweetener. On the other hand, some embodiments of the present invention do not include any cariogenic sweetener. This is because the inclusion of some cariogenic sweeter may actually increase the formation of bacteria-related caries. In any event, some examples can include monosaccharides, disaccharides, polysaccharides of ribose, glucose, mannose, galactose, fructose, dextrose, sucrose, and sugar maltose. If used, it is preferred that compositions of the present invention comprise less than about 5%, preferably less than about 2%, more preferably less than about 1%, even more preferably less than about 0.5%, and most preferably no cariogenic sweetener.

Flavoring agents other than sweeteners can also be included in the oral hygiene composition. Flavoring agents are well known in the art, and include synthetic flavors and/or oils and/or essences derived from plants, roots, beans, nuts, leaves, flowers, fruits and the like. Some examples of suitable flavors include lemon, orange, banana, grape, lime, apricot, grapefruit, apple, strawberry, cherry, chocolate, pineapple, coffee, cocoa, cola, peanut, almond, liquorices, cinnamon and the like. The amount of flavoring agent employed is a matter of preference, but can be used up to about 5% by weight of the composition.

Also, the flavorant can be considered to be a cooling agent. Some examples of substances that can be considered physiological cooling agents include menthol, peppermint oil, acyclic tertiary and secondary carboxamides, 3-1-methoxy propan-1,2-diol and the like. When used, cooling agents can be included at from about 0.001% to about 5% by weight of the composition.

The oral hygiene composition of the present invention may also include a polyoxyethylene also referred to as polyethylene glycol (“PEG”). The polyoxyethylene can increase the amount of foam and the thickness of the foam generated after the composition is applied to a tooth or gingiva. The polyoxyethylenes suitable for this invention have a molecular weight of about 200,000 to about 7,000,000. In one embodiment the molecular weight will be from about 600,000 to about 2,000,000, and in another embodiment from about 800,000 to about 1,000,000. The polyoxyethylene may be present in the composition at from about 0. 1% to about 8%, preferably from about 0.2% to about 5%, and more preferably from about 0.3% to about 2% by weight.

Chemical enhancers can also be included in the present compositions. This is because an enhancer can increase the ability of erythritol to enter the gingiva around a site where an infant's teeth are forming. This can be especially useful for cleansing during teething. Some examples of enhancers can include saturated and unsaturated fatty acids and their esters, alcohols, monoglycerides, acetates, diethanolamides, and N,N-dimethylamides, such as oleic acid, propyl oleate, isopropyl myristate, glycerol monooleate, glycerol monolaurate, methyl laurate, lauryl alcohol, lauramide diethanolamide, and the like. Saturated and unsaturated sorbitan esters, such as sorbitan monooleate and sorbitan monolaurate, can also be used, among others.

An abrasive polishing material may also be included in the oral hygiene composition. The abrasive polishing material can be any material which does not abrade dentin or gingiva, but acts to polish a tooth or remove tarter. Some examples of polishing agents includes silica gels and precipitates, aluminates, phosphates, and mixtures thereof. Some specific examples include, without limitation, silica, silicate, aluminosilicate, calcium phosphate, dicalcium orthophosphate dihydrate, calcium pyrophosphate, tricalcium phosphate, hydrated alumina, beta calcium pyrophosphate, calcium carbonate, and the like. Mixtures of abrasives may also be used.

For example, the abrasive polishing materials such as silica can have an average particle size ranging between about 0.1 to about 50 microns, and preferably from about 5 to about 15 microns. When present, the abrasive in the compositions herein is generally present at a level of from about 1 % to about 70%, or from about 10% to about 50%, by weight of the composition.

Additionally, an embodiment of the present invention is an oral hygiene composition that can be diluted with water or other solution prior to being delivered to the teeth and gums. Accordingly, in order for the composition to have favorable storage characteristics, the composition can be formulated to be concentrated so that it can be diluted into a saline or aqueous solution prior to administration. Also, the composition can be formulated into a solid or powder so that it can be dissolved in water or a saline solution prior to administration. Accordingly, the composition can be processed into a liquid, suspension, paste, gel, powder, or solid, where such a composition can be administered as provided or diluted as desired or needed to facilitate administration.

Another embodiment of the present invention is a method of oral cleansing. Such a method can be performed in a subject incapable of self-performing dental hygiene techniques. Examples of subjects incapable of self-performing dental hygiene techniques includes infants, toddlers, quadriplegics, and other handicapped subjects as well as any person that cannot sufficiently brush their teeth, floss, and/or gargle with mouthwash.

The method of oral cleansing can include administering an erythritol-containing composition in an effective amount to the teeth or gingiva. Such an effective amount includes a quantity that can induce any of the physiological processes described herein.

Another embodiment of a method includes administering an oral hygiene composition to one of the subject's tooth and gingiva in an effective amount. Accordingly, the oral hygiene composition includes an effective amount of erythritol admixed into an acceptable carrier. Additionally, the acceptable carrier, along with the oral hygiene composition can be configured into a gel.

The method can also include retaining the oral hygiene composition to the one of the tooth and gingiva for a sufficient duration. Retaining some portion of the composition at the site of administration provides for enhancing the beneficial effect. This is because the longer the composition can be retained on the gingiva or teeth, the longer the composition can supply erythritol thereto. Accordingly, after the composition is administered, the subject may close their mouth or lick the composition, which may dissolve or remove some of the composition. However, configuring the composition to have at least a portion be retained to the site of administration can be effected by configuring the composition to have some bioadhesive characteristics, which can be obtained by the oral hygiene composition disclosed herein.

Additionally, a method in accordance with the present invention can include administering the erythritol-containing composition in an amount effective at preventing and/or treating dental caries. Accordingly, the amount can be effect for inhibiting the amount of acid produced by bacteria. More particularly, the amount can be effective at decreasing the amount of acid produced by strep mutan or other bacteria. Also, the amount can be effective at inhibiting the growth of bacteria, especially strep mutans. It is thought, without being bound to theory, that the erythritol-containing composition can inhibit the growth of bacteria by interfering with the bacteriological life cycle, which includes inhibiting bacteria cell growth and inhibiting bacteria reproduction.

Additionally, by inhibiting bacteria growth or bacteria acid production, the erythritol-containing composition can produce various beneficial effects in the mouth, especially with respect to the teeth and gingiva. Accordingly, a method in accordance with the present invention can further include inhibiting the formation of dental caries. Since the formation of dental caries can be linked at least partially to bacteria, erythritol can reduce the instances and formation of dental caries. Similarly, a method can include reducing a bacterial film on one of the teeth and gingiva.

A method of the present invention can also include reducing plaque buildup on the subject's tooth. Also, a method can include preventing and/or reducing tartar or calculus buildup on the teeth. Accordingly, the process of removing plaque or tartar may at least partially occur because of the anti-bacteriological effect of the erythritol-containing composition.

In related embodiments of the present invention, a method of administering the erythritol-containing composition can be performed before, during, and/or after teething. As such, the erythritol-containing composition can be used in a method for reducing teething-associated inflammation or soreness. During teething, infants can exhibit various emotional manifestations in reaction to the pain associated therewith. These emotional displays may act to prevent a parent or other care giver from administering the erythritol-containing composition during teething. In order to be able to administer the erythritol-containing composition during teething, it may be beneficial for a parent or care giver to train the infant to accept and administration instead of fighting it. This can be done by administering the composition to the gingiva long before the process of teething actually occurs. This can also be aided by the sweetness of erythritol, where, if properly trained or conditioned, the infant may look forward to the administration. By getting an infant acclimated to daily erythritol administrations, they may be less likely to resist such applications during teething. Also, conditioning administrations in infants can also aid in effecting the cleansing and anti-bacteriological properties of erythritol.

Additionally, the administering of erythritol-containing compositions can be performed by any technique that places some portion of the composition on the teeth or gingiva. Some of the methods of administration can include directly applying the erythritol-containing composition to the teeth and gingiva from a container. Accordingly, such a container can be configured to include an orifice that facilitates such a direct application. Also, the container can be squeezable so that quantity administered can be metered and/or evenly distributed to the sites of application. The various containers that can effect such administration are well known in the art.

The container can also be used to apply the erythritol-containing composition to an applicator. When used, the applicator then can be used to administer the erythritol-containing composition to the teeth and gingiva. Some examples of applicators can include cotton tip applicator, toothbrush, gauze, pad, and floss.

The quantity of the erythritol-containing composition can be varied depending on various factors. Such factors include the size of the subject mouth, gingiva, teeth, and the like. Also, the quantity can be varied by the method of administration. In any event, the beneficial properties of the erythritol-containing compositions can be realized by consistent use.

For daily cleansing, or during any application, the administering can include applying a unit dose form in a sufficient amount of the erythritol-containing composition to result in a beneficial purpose. In some embodiments, the amount or unit dose form can be at least one drop of the composition such as one drop of gel. For example, the amount can be between about 2 to about 5 drops per application. Also, the amount can include a line of gel along the gingiva, in front of the teeth, and/or behind the teeth. Accordingly, the amount can be increased or decreased as needed.

Also, the administering can be part of a therapeutic regimen to provide any of the beneficial purposes described herein. Such a regimen can vary depending on the desired results as well as the compositional elements in the formulation being administered. The regimen can include from one administration per day to multiple administrations per day such as, for example, between 3 to 5 applications per day. Alternatively, the administering regimen can be correlated to various activities that commonly occur. Some examples of correlations with various activities includes after eating or drinking, at the time of changing a diaper, before sleeping, after awaking, and the like.

For example, a regimen can begin at or before 6 months of age, where the erythritol-containing composition can be applied on the gingiva before or during teething, and/or on the infant's or toddler's teeth after teething. From 6-18 months, 2-5 drops of the erythritol-containing composition such as the erythritol-gel can be applied to the gums and/or teeth with a cotton tip applicator. From 18 months to 3 years, 2-5 drops of the erythritol-containing composition can be applied on a toothbrush and brushed onto the teeth. In any of these regimens, the erythritol-containing composition can be applied 3 to 5 times daily after feedings, meals, snacks, and/or at bedtime.

When used regularly, the erythritol-containing composition can reduce cavities by up to 80%. The erythritol-containing compositions in accordance with the present invention can be used to controlling the strep mutans bacteria in the mouth, thus lowering the chances of tooth decay. Additionally, the erythritol-containing composition can reduce the gingiva tissue inflammation that occurs during teething. With the present inventive compositions, parents now have a safe and effective solution to help their babies and toddlers who are just cutting teeth to benefit from the unique qualities of erythritol.

A more specific example of a method in accordance with the present invention can include administering an oral hygiene composition to one of the child's tooth and gingiva in an effective quantity, where the oral hygiene composition includes a therapeutically effective amount of erythritol admixed into a pharmaceutically acceptable carrier. Accordingly, the pharmaceutically acceptable carrier can include at least one component selected from the group consisting of thickening or gelling agent, calcium agent, preservative, humectant, flavorant, abrasive agent, and desensitizing agent, wherein the pharmaceutically acceptable carrier is configured into a gel. Additionally, the method includes retaining the oral hygiene gel to the one of the tooth and gingiva for a sufficient duration so that the erythritol can act as an agent for inhibiting one of bacteria acid production and bacteria growth, and inhibiting the formation of dental caries. The oral hygiene gel can be formulated to include about 10% to about 80% erythritol; about 15% to about 50% gelling agent, the gelling agent being selected from the group consisting of polysaccharides, celluloses, carboxymethylcelluloses, starches, amyloses, amylopectins, glycogens, chitins, chitosans, agars, carrageenans, guars, pectins, xanthans, glucomannan gums, and combinations thereof; about 1% to about 50% calcium agent, the calcium agent being selected from the group consisting of calcium acetate, calcium citrate, calcium gluconate, calcium glycerol phosphate mono-calcium phosphate, mono-calcium phosphate anhydrous, di- and tri-calcium phosphate, calcium sulfate, calcium carbonate, calcium lactate, calcium phosphate, and combinations thereof, and about 10% to about 60% water.

The present invention is further exemplified in the following examples, which are offered by way of illustration and are not intended to limit the invention in any manner.

### EXAMPLES

### Example 1

### Erythritol Gel

An erythritol-containing oral hygiene gel is formulated in accordance with the present invention. The gel is prepared by mixing 15% erythritol, 30% cellulose gum, 10% calcium glycerol phosphate, and 44.8% purified water, where all percentages represent the amount per weight of the final gel. Additionally, grapefruit seed extract is added as a preservative at 0.2% of the gel. The mixture is then mixed so that the components become homogeneously distributed with one another to form a gel, which can be denoted as Formulation 1. The gel is then loaded into a squeeze-container so that it can be administered drop-wise.

### Example 2

### Erythritol Compositions

Erythritol compositions containing various components and associated concentrations are prepared in accordance with the procedure described in Example 1. The resulting erythritol compositions are illustrated in Table 1.

TABLE 1

| | |
|---|---|
| Component | % (by weight) |

FORMULATION 2

| | |
|---|---|
| Erythritol | 25 |
| Carboxymethylcellulose (15,000 Mw) | 2 |
| Calcium glycerol phosphate | 6.5 |
| Methyl laurate | 5 |
| Menthol | 1 |
| Purified water | 60.5 |

FORMULATION 3

| | |
|---|---|
| Erythritol | 25 |
| Xylitol | 5 |
| Carboxymethylcellulose (15,000 Mw) | 12 |
| Calcium glycerol phosphate | 2.5 |
| Lauryl alcohol | 5 |
| Propylene glycol | 5 |
| Purified Water | 45.5 |

FORMULATION 4

| | |
|---|---|
| Erythritol | 35 |
| Carboxymethylcellulose (15,000 Mw) | 21 |
| Calcium glycerol phosphate | 6.5 |
| Phosphoric acid | 1 |
| Sodium bicarbonate | 1.5 |
| Purified Water | 35 |

FORMULATION 5

| | |
|---|---|
| Erythritol | 40 |
| Carboxymethylcellulose (15,000 Mw) | 25 |
| Calcium glycerol phosphate | 4 |
| Calcium phosphate | 1 |
| Purified Water | 30 |

FORMULATION 6

| | |
|---|---|
| Erythritol | 45 |
| Carboxymethylcellulose (15,000 Mw) | 20 |
| Gum arabic | 1 |
| Calcium glycerol phosphate | 5 |
| Castor oil | 1 |
| Purified Water | 28 |

FORMULATION 7

| | |
|---|---|
| Erythritol | 50 |
| Carboxymethylcellulose (15,000 Mw) | 20 |
| Calcium glycerol phosphate | 5 |
| Cyclodextran | 1 |
| Purified Water | 24 |

FORMULATION 8

| | |
|---|---|
| Erythritol | 55 |
| Carboxymethylcellulose (15,000 Mw) | 15.5 |
| Calcium glycerol phosphate | 5 |
| Menthol | 0.5 |
| Purified Water | 24 |

FORMULATION 9

| | |
|---|---|
| Erythritol | 35 |
| Ticalose ® (TIC Gums, Belcamp, MD) | 2.5 |
| Calcium | 1.5 |
| Grapefruit seed extract | <0.1 |
| Purified Water | ˜61 |

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. An oral hygiene composition for use in cleansing a subject's teeth and gingiva, the composition comprising:
an effective amount of erythritol that is capable of cleansing the subject's teeth or gingiva; and
an acceptable carrier admixed with the effective amount of erythritol, wherein the acceptable carrier is configured into a composition that is capable of being applied to at least one of a subject's tooth or gingiva in a sufficient amount so as to be retained thereto for a duration adequate for cleansing the subject's teeth or gingiva.

2. A composition as in claim 1, wherein the erythritol is present at a concentration from about 10% to about 80% by weight of the composition.

3. A composition as in claim 2, wherein the carrier includes a thickening agent.

4. A composition as in claim 3, wherein the carrier includes calcium agent.

5. A composition as in claim 4, wherein the carrier includes a component selected from the group consisting of preservatives, humectants, polishing agents, acidic agents, buffers, enhancers, flavorants, softeners, plasticizers, and emulsifiers, and desensitizing agents, malodor agents, and non-cariogenic sweeteners, wherein the composition is substantially devoid of a cariogenic sweetener.

6. A method of oral cleansing in a subject incapable of adequately self-performing dental hygiene techniques, the method comprising:
administering a oral hygiene composition to at least one of the subject's tooth or gingiva, the oral hygiene composition including an effective amount of erythritol admixed into an acceptable carrier; and
retaining the oral hygiene composition to the at least one of the tooth or gingiva for a sufficient duration to effect the oral cleansing.

7. A method as in claim 6, wherein the method includes inhibiting at least one of bacteria acid production or bacteria growth.

8. A method as in claim 7, wherein the method includes inhibiting formation of dental caries.

9. A method as in claim 6, wherein the method includes reducing plaque buildup on the subject's tooth.

10. A method as in claim 7, wherein the method includes reducing a bacterial film on one of the teeth and gingiva.

11. A method as in claim 6, wherein the method includes reducing teething-associated inflammation or soreness.

12. A method as in claim 7, wherein the inhibiting occurs during or after teething.

13. A method as in claim 6, wherein the administering delivers an effective quantity to effect the oral cleansing

14. A method as in claim 13, wherein the oral hygiene composition is a gel.

15. A method as in claim 6, wherein the administering is by at least one of a cotton tip applicator, toothbrush, gauze, pad, or gel dispenser.

16. A method as in claim 15, wherein the sufficient amount includes at least one drop of the gel.

17. A method as in claim 16, wherein the administering is part of a therapeutic regimen that includes at least 3 separate acts of administering per day.

18. A method as in claim 15, wherein the administering occurs after eating or before sleeping.

19. A method of treating and/or preventing dental caries in a child, the method comprising:
administering a oral hygiene composition to at least one of the child's tooth or gingiva in an effective amount, the oral hygiene composition including erythritol admixed into an acceptable carrier, the acceptable carrier including at least one component selected from the group consisting of gelling agent, calcium agent, preservative, humectant, flavorant, abrasive agent, non-cariogenic sweetener, and desensitizing agent, wherein the acceptable carrier is configured into a gel;
retaining the oral hygiene composition to the at least one of the tooth or gingiva for a sufficient duration; and
inhibiting at least one of bacteria acid production or bacteria growth.

20. A method as in claim 19, wherein the oral hygiene composition comprises:
about 10% to about 80% erythritol;
about 1% to about 50% gelling agent, the gelling agent being selected from the group consisting of polysaccharides, celluloses, carboxymethylcelluloses, starches, amyloses, amylopectins, glycogens, chitins, chitosans, agars, carrageenans, guars, pectins, xanthans, glucomannan gums, and combinations thereof; and
about 1% to about 5% calcium agent, the calcium agent being selected from the group consisting of calcium, calcium acetate, calcium citrate, calcium gluconate, calcium glycerol phosphate, mono-calcium phosphate, mono-calcium phosphate anhydrous, di- and tri-calcium phosphate, calcium sulfate, calcium carbonate, calcium lactate, calcium phosphate, and combinations thereof.
